(19) United States
(12) Patent Application Publication
(10) Pub. No.: US 2008/0119740 A1
(43) Pub. Date: May 22, 2008

(54) Device, System, and Method for Optical In-Vivo Analysis

(76) Inventor: Gavriel J. Iddan, Haifa (IL)

Correspondence Address: Pearl Cohen Zedek Latzer, LLP, 1500 Broadway, 12th Floor, New York, NY, 10036, US

(21) Appl. No.: 11/794,423

(22) PCT Filed: Dec. 28, 2005
(86) PCT No.: PCT/IL05/01394

§ 371 Date: Jun. 28, 2007

Related U.S. Application Data
(60) Provisional application No. 60/639,965, filed on Dec. 30, 2004.

(51) Int. Cl. A61B 6/00

(52) U.S. Cl. 600/477; 356/300

(57) ABSTRACT

Devices, systems and methods for in-vivo imaging include, for example, a separator configured to separate a light, reflected from a body lumen, into at least one separated-color light, and optionally an analyzer configured to analyze the at least one separated-color light.

### FIELD OF THE INVENTION

The present invention relates to the field of imaging. More specifically, the present invention relates to devices, systems, and methods for in-vivo imaging.

### BACKGROUND OF THE INVENTION

Devices, systems and methods for in-vivo imaging of passages or cavities within a body are known in the art.

An in-vivo imaging system may include, for example, an in-vivo imaging device for obtaining images from inside a body cavity or lumen, such as the gastrointestinal (GI) tract. The in-vivo imaging device may include, for example, an imager associated with units such as, for example, an illumination source, a controller or processor, a power source, a transmitter, and an antenna. Other types of in-vivo devices exist, such as endoscopes which may not require a transmitter, and in-vivo devices performing functions other than imaging.

Some in-vivo imaging devices illuminate a body organ, tissue or lumen using an illumination source, acquire the reflected light using an imager, and transmit image data using a transmitter to an external receiver.

### SUMMARY OF THE INVENTION

Various embodiments of the invention provide, for example, an in-vivo imaging device and a method and system for using the in-vivo imaging device.

In accordance with some embodiments of the invention, the in-vivo imaging device may include, for example, a separator or other unit configured to separate a light, reflected from a body lumen, into at least one separated-color light; and optionally an analyzer configured to analyze said at least one separated-color light.

In accordance with some embodiments of the invention, the separator may include, for example, a prism, a grating, a color filter or a color divider.

In accordance with some embodiments of the invention, the separator may include, for example, an optical system having a Michelson Interferometer arrangement, and the analyzer may be configured to analyze an interference pattern produced in the in-vivo imaging device.

In accordance with some embodiments of the invention, the analyzer may be configured to analyze at least one separated-color light in relation to a pre-defined reference value. In accordance with some embodiments of the invention, the analyzer may be configured to determine whether at least one separated-color light is reflected from a healthy tissue, or a non-healthy tissue.

In accordance with some embodiments of the invention, the in-vivo imaging device may include, for example, an illumination unit to illuminate a white light through a non-dispersive lens; a semi-reflective mirror or other beam splitter to split said white light into substantially a first half and a second half, said first half illuminated onto said body lumen, and said second half reflected back towards said illumination unit; a dispersive lens to receive said first half reflected from said body lumen; and a non-dispersive lens to focus an interference pattern of said first half transferred through said dispersive lens and said second half.

In accordance with some embodiments of the invention, the separator may include, for example, a panoramic mirror to receive and reflect the light reflected from the body lumen; and a grating to separate the light reflected from said panoramic mirror into at least one separated-color light.

In accordance with some embodiments of the invention, the in-vivo imaging device may include, for example, an illumination unit to illuminate a substantially ring-shaped white light or dispersion of white light towards the body lumen.

In accordance with some embodiments of the invention, the in-vivo imaging device may be, for example, a swallowable capsule and/or an autonomous in-vivo device.

In accordance with some embodiments of the invention, the in-vivo imaging device may be used, for example, to perform separation of light reflected from a body lumen, e.g., into separate color lights, and analysis of one or more of the separate color lights.

Embodiments of the invention may allow various other benefits, and may be used in conjunction with various other applications.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a schematic illustration of an in-vivo imaging system in accordance with an embodiment of the invention;

FIG. 2 is a schematic illustration of an in-vivo imaging device in accordance with an embodiment of the invention;

FIG. 3 is a schematic illustration of an in-vivo imaging device in accordance with another embodiment of the invention;

FIG. 4 is a schematic illustration of an in-vivo imaging device in accordance with yet another embodiment of the invention; and

FIG. 5 is a flow-chart diagram of a method in accordance with an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

While a portion of the discussion may relate, for exemplary purposes, to features, functionalities and components of an in-vivo imaging device, embodiments of the present invention are not limited in this regard, and may be used not necessarily in the context of in-vivo imaging.

Some embodiments of the present invention are directed to a typically swallowable in-vivo device, e.g., a typically swallowable in-vivo sensing or imaging device. Devices according to embodiments of the present invention may be similar to embodiments described in U.S. patent application Ser. No. 09/800,470, entitled “Device and System for In-vivo Imaging”, filed on 8 Mar., 2001, published on Nov. 1, 2001 as United States Patent Application Publication Number 2001/0035902, and/or in U.S. Pat. No. 5,604,531 to Iddan et al., entitled “In-Vivo Video Camera System”, and/or in U.S. patent application Ser. No. 10/046,541, filed on Jan. 16, 2002, published on Aug. 15, 2002 as United States Patent Application Publication Number 2002/0109774, all of which are hereby incorporated by reference. An external receiver/recorder unit, a processor and a monitor, e.g., in a workstation, such as those described in the above publications, may be suitable for use with some embodiments of the present invention. Devices and systems as described herein may have other configurations and/or other sets of components. For example, some embodiments of the present invention may be practiced using an endoscope, needle, stent, catheter, etc. Some in-vivo devices may be capsule shaped, or may have other shapes, for example, a peanut shape or tubular, spherical, conical, or other suitable shapes. For example, an optical biopsy according to an embodiment of the invention may be utilized in probes used for in vivo imaging, such as endoscopes.

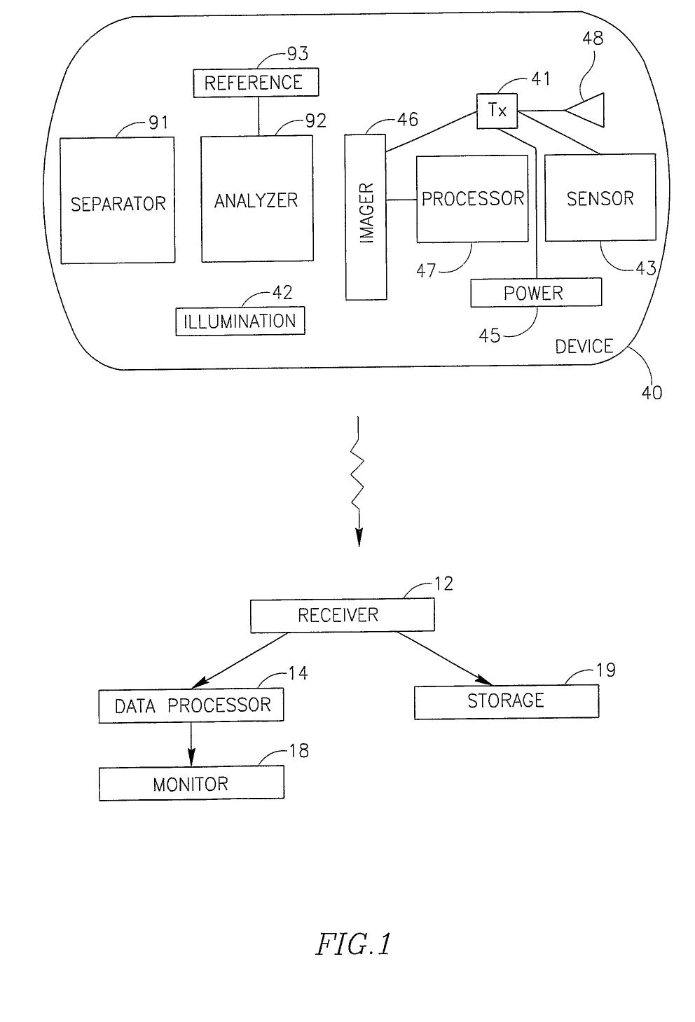

FIG. 1 shows a schematic diagram of an in-vivo imaging system in accordance with an embodiment of the present invention. In one embodiment, for example, the system may include a device 40 having an imager 46, one or more illumination sources 42, a power source 45, and a transmitter 41. In some embodiments, for example, device 40 may be implemented using a swallowable capsule, or may be inserted into a patient's body by for example swallowing, but other sorts of devices or implementations or methods of insertion may be used. Outside a patient's body may be, for example, an image receiver 12 (including, for example, an antenna or an antenna array), a storage unit 19, a data processor 14, and a monitor 18.

Transmitter 41 may operate using radio waves; but in some embodiments, such as those where device 40 is or is included within an endoscope, transmitter 41 may transmit data via, for example, wire, optical fiber and/or other suitable methods.

Device 40 typically may be or may include an autonomous swallowable capsule, but device 40 may have other shapes and need not be swallowable and/or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power or transmit information.

In some embodiments, device 40 may be a swallowable capsule shaped and able to operate as an autonomous video endoscope for imaging, for example, the GI tract. However, other devices, such as devices designed to be incorporated in an endoscope, catheter, stent, needle, etc., may also be used according to embodiments of the invention. Furthermore, device 40 need not include all the elements or components described herein. For example, in one embodiment, device 10 need not include internal illumination source 42 or internal power source 45; illumination and/or power may be provided using an external source or another in vivo device.

In some embodiments, device 40 may communicate with an external receiving and display system (e.g., through receiver 12) to provide display of data, control, or other functions. For example, power may be provided to device 40 using an internal battery, an internal power source, or a wireless system to receive power. Other embodiments may have other configurations and capabilities. For example, components may be distributed over multiple sites or units, and control information may be received from an external source.

In one embodiment, device 40 may include an in-vivo video camera or light sensor, for example, an image sensor or imager 46, which may capture and transmit images of, for example, the GI tract while device 40 passes through the GI lumen. Other lumens and/or body cavities may be imaged and/or sensed by device 40. In some embodiments, imager 46 may include, for example, a Charge Coupled Device (CCD) camera or imager, a Complementary Metal Oxide Semiconductor (CMOS) camera or imager, a digital camera, a stills camera, a video camera, or other suitable imagers, cameras, or image acquisition components. In some embodiments, imager 46 may include, for example, a light sensor, a light detector, a color sensor or a color detector (e.g., including or using one or more diodes or other suitable components), and may include but need not necessarily include an image acquisition unit.

In one embodiment, imager 46 in device 40 may be operationally connected to transmitter 41. Transmitter 41 may transmit images to, for example, image receiver 12, which may send the data to data processor 14 and/or to storage unit 19. Transmitter 41 may also include control capability, although control capability may be included in a separate component. Transmitter 41 may include any suitable transmitter able to transmit image data, other sensed data, and/or other data (e.g., control data) to a receiving device. For example, transmitter 41 may include an ultra low power Radio Frequency (RF) high bandwidth transmitter, possibly provided in Chip Scale Package (CSP). Transmitter 41 may transmit via antenna 48. Transmitter 41 and/or another unit in device 40, e.g., a controller or processor 47, may include control capability, for example, one or more control modules, processing module, circuitry and/or functionality for controlling device 40, for controlling the operational mode or settings of device 40, and/or for performing control operations or processing operations within device 40.

Power source 45 may include one or more batteries or power cells. For example, power source 45 may include silver oxide batteries, lithium batteries, other suitable electrochemical cells having a high energy density, or the like. Other suitable power sources may be used. For example, power source 45 may receive power or energy from an external power source (e.g., a power transmitter), which may be used to transmit power or energy to device 40.

Optionally, in one embodiment, transmitter 41 may include a processing unit or processor or controller, for example, to process signals and/or data generated by imager 46. In another embodiment, the processing unit may be implemented using a separate component within device 40, e.g., controller or processor 47, or may be implemented as an integral part of imager 46, transmitter 41, or another component, or may not be needed. The optional processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In one embodiment, for example, the processing unit or controller may be embedded in or integrated with transmitter 41, and may be implemented, for example, using an ASIC. In some embodiments, one or more processing operations may be performed by or more separate units, within device 40 and/or external to device 40, e.g., in a workstation or data processor 14.

In some embodiments, imager 46 may acquire in-vivo images continuously, substantially continuously, or in a non-discrete manner, for example, not necessarily upon-demand, or not necessarily upon a triggering event or an external activation or external excitement; or in a periodic manner, an intermittent manner, or an otherwise non-continuous manner. In some embodiments, transmitter 41 may transmit image data continuously, or substantially continuously, for example, not necessarily upon-demand, or not necessarily upon a triggering event or an external activation or external excitement; or in a periodic manner, an intermittent manner, or an otherwise non-continuous manner.

In some embodiments, device 40 may include one or more illumination sources 42, for example one or more Light Emitting Diodes (LEDs), “white LEDs”, or other suitable light sources. Illumination sources 42 may, for example, illuminate a body lumen or cavity being imaged and/or sensed.

Data processor 14 may analyze the data received via receiver 12 from device 40, and may be in communication with storage unit 19, e.g., transferring frame data to and from storage unit 19. Data processor 14 may also provide the analyzed data to monitor 18, where a user (e.g., a physician) may view or otherwise use the data. In one embodiment, data processor 14 may be configured for real time processing and/or for post processing to be performed and/or viewed at a later time. In the case that control capability (e.g., delay, timing, etc) is external to device 40, a suitable external device (such as, for example, data processor 14 or image receiver 12) may transmit one or more control signals to device 40.

Monitor 18 may include, for example, one or more screens, monitors, or suitable display units. Monitor 18, for example, may display one or more images or a stream of images captured and/or transmitted by device 40, e.g., images of the GI tract or of other imaged body lumen or cavity. Additionally or alternatively, monitor 18 may display, for example, control data, location or position data (e.g., data describing or indicating the location or the relative location of device 40), orientation data, and various other suitable data. In one embodiment, for example, both an image and its position or location may be presented using monitor 18 and/or may be stored using storage unit 19. Other systems and methods of storing and/or displaying collected image data and/or other data may be used.

In some embodiments, in addition to or instead of revealing pathological or other conditions of the GI tract, the system may provide information about the location of these conditions. Suitable tracking devices and methods are described in embodiments of the above-mentioned U.S. Pat. No. 5,604,531 and/or United States Patent Application Publication Number US-2002-0173718, filed on May 20, 2002, titled “Array System and Method for Locating an In-Vivo Signal Source”, assigned to the common assignee of the present invention, and fully incorporated herein by reference. Other suitable location identification systems and methods may be used in accordance with embodiments of the present invention.

Typically, device 40 may transmit image information in discrete portions. Each portion may typically correspond to an image or a frame; other suitable transmission methods may be used. For example, in some embodiments, device 40 may capture and/or acquire an image once every half second, and may transmit the image data to receiver 12. Other constant and/or variable capture rates and/or transmission rates may be used.

Typically, the image data recorded and transmitted may include digital color image data; in alternate embodiments, other image formats (e.g., black and white image data) may be used. In one embodiment, each frame of image data may include 256 rows, each row may include 256 pixels, and each pixel may include data for color and brightness according to known methods. According to other embodiments, a 320 by 320 pixel imager may be used. Pixel size may be, for example, between 5 to 6 microns. According to some embodiments, pixels may be each fitted with a micro lens.

In some embodiments, for example, in each pixel, color may be represented by a mosaic of four sub-pixels, each sub-pixel corresponding to primaries such as red, green, or blue (where one primary, e.g., green, may be represented twice). The brightness of the overall pixel may be recorded by, for example, a one byte (e.g., 0-255) brightness value. In one embodiment, for example, image data may be represented using an array of 64 by 64 pixels or super-pixels or boxes, each including data indicating values for red, green (repeated twice) and blue. Other suitable data formats may be used, and other suitable numbers or types of rows, columns, arrays, pixels, sub-pixels, boxes, super-pixels and/or colors may be used.

In some embodiments, image data may be, but need not be, transmitted by device 40. For example, in one embodiment, instead or in addition to image data, device 40 may transmit color data, color levels data, light levels data, hue data, saturation data, brightness data, spectral data, or the like. In another embodiment, instead or in addition to image data, device 40 may transmit data indicating one or more results from an analysis of an acquired image, or from an analysis of color data, color levels data, light levels data, hue data, saturation data, brightness data, spectral data, or the like. In some embodiments, color separation data or separated-color data may be transmitted by device 40, instead of or in addition to image data, and/or substantially simultaneously or together with image data. In some embodiments, for example, device 40 may transmit image data corresponding to color separation data or separated-color data.

Optionally, device 40 may include one or more sensors 43, instead of or in addition to a sensor such as imager 46. Sensor 43 may, for example, sense, detect, determine and/or measure one or more values of properties or characteristics of the surrounding of device 40. For example, sensor 43 may include a pH sensor, a temperature sensor, an electrical conductivity sensor, a pressure sensor, or any other known suitable in-vivo sensor.

In one embodiment, device 40 may include one or more units to perform an optical biopsy in vivo by device 40, as described herein. In another embodiment, device 40 may include one or more units to allow performing an optical biopsy by a combination of device 40 and/or an external device or system, e.g., using data processor 14.

In accordance with some embodiments of the invention, white light may be illuminated in-vivo, e.g., towards a body organ or lumen, for example, by illumination source(s) 42. White light includes a combination or blend of substantially all colors of the spectrum, e.g., red, orange, yellow, green, blue, indigo and violet. In accordance with some embodiments of the invention, different colors may be reflected from the body organ or lumen in different reflection patterns, in different light strengths or intensities, from different depths, or in accordance with different reflection characteristics or properties. In some embodiments, white light need not be used; in some embodiments, light or lights other than white light may be used, instead or in addition to white light.

For example, in some embodiments, a blue light may be reflected by a first layer or a body tissue, and may not penetrate deep into the body tissue, whereas a red light may be reflected by a second, deeper layer of a body tissue, as the red light may penetrate deeper into the body tissue relative to the penetration of the blue light. For example, light rays or light beams having different colors may be able to penetrate into different depths, e.g., in an order of magnitude of micrometers or other sub-millimeter units. In accordance with some embodiments, white or other light may be illuminated in-vivo towards a body organ or lumen, and the reflected light may be separated into colors for analysis, e.g., in relation to pre-defined reference values, for example, to determine or estimate one or more properties of the body organ or lumen that reflected the light.

In some embodiments, analysis of processing of separated light may allow, for example, more accurate or more sensitive results, e.g., relative to a calorimetric analysis or processing of full-color images or Red Green Blue (RGB) image data.

In some embodiments, device 40 may include a color separator 91, for example, a grating, a prism, one or more filters, a color divider, a lens or lens assembly, a mirror, a reflective or semi-reflective optical element, a refractive optical element, a defractive optical element, or other suitable one or more optical element. In some embodiments, the illuminated white or other light may be reflected from the body organ or lumen, and may be received by separator 91 which may separate or divide the light into one or more colors, lights, wavelength, bandwidths, spectra, or spectral elements. For example, the reflected light may be separated by separator 91 into a blue light, a red light, an orange light, and/or other colors of the spectrum, e.g., if such color lights exist in the reflected light.

In some embodiments, a light having one or more of the separated colors may be received by a detector or analyzer unit 92. In one embodiment, analyzer 92 may include an imager similar or identical to imager 46, e.g., in addition to imager 46. In another embodiment, analyzer 92 may be implemented as an integrated unit or sub-unit of imager 46, or as a section (e.g., a corner area, a side area or a center area) of imager 46. In yet another embodiment, analyzer 92 may include an optical detection system or one or more optical sensor(s), which need not necessarily include an imager or an image acquisition unit, and may be able to detect and/or analyze one or more pre-defined colors of light, wavelengths, bandwidths, or spectra. In still another embodiment, analyzer 92 may include one or more light-sensitive diodes or color-sensitive diodes, e.g., a first diode sensitive to red color, a second diode sensitive to green color, and a third diode sensitive to blue color. In some embodiments, analyzer 92 may generate one or more values responsive to the light received, e.g., values indicating a color level value, a level value of one or more colors, or other suitable values.

In some embodiments, analyzer 92 may optionally include one or more optical filters, e.g., a filter to allow transfer of light rays having one or more pre-defined colors and to block light rays not having said one or more pre-defined colors, e.g., a blue color filter or a red color filter.

In some embodiments, analyzer 92 may sense one or more properties of the received light. For example, analyzer 92 may include a detector, a sensor, and imager or an analyzing unit able to receive and detect blue light, and able to analyze or process one or more properties of the received blue light, e.g., light strength or intensity, angle of reception, pattern or focus of the received light, or other characteristic. In some embodiments, analyzer 92 may include a processing unit similar to processor 47, or may be integrated with processor 47, to allow analyzer 92 to perform the analysis.

In some embodiments, device 40 or data processor 14 may optionally include a reference value storage unit 93, for example, a memory unit able to store one or more pre-defined reference values which may be used by analyzer 92. In one embodiment, storage unit 93 may include, for example, a Random Access Memory (RAM), a Dynamic RAM (DRAM), a Synchronous DRAM (SD-RAM), a Flash memory, a volatile memory, a non-volatile memory, a modifiable memory, a programmable memory, a writeable memory, a cache memory, a buffer, one or more registers, one or more lookup tables, one or more tables, one or more maps or mapping tables, a short term memory unit, a long term memory unit, or other suitable memory units or storage units.

For example, in one embodiment, it may be pre-determined that a blue light typically reflected from a healthy tissue has a first value (“reference value”) of strength or intensity, and this first value may be stored in storage unit 93. Analyzer 92 may determine the value of the strength or intensity of the blue light received from separator 91 (“sensed value”), and may compare the sensed value to the reference value stored in the storage unit 93.

In one embodiment, analyzer 92 may determine that the sensed value is substantially equal to the reference value, and may thereby determine and/or indicate that the blue light was reflected from a healthy tissue. In another embodiment, analyzer 92 may determine that the sensed value if substantially different (e.g., smaller or higher) than the reference value, and may thereby determine and/or indicate that the blue light was reflected from a non-healthy tissue, e.g., from an abnormal tissue or a tissue having a pathology. In yet another embodiment, analyzer 92 may analyze the sensed value in relation to one or more reference values, for example, a maximum threshold value, a minimum threshold value, a range of values, or the like.

In one embodiment, a reference value stored in storage unit 93 may relate to a healthy tissue, such that analyzer 92 may process the sensed value in relation to a reference value of a healthy tissue. In another embodiment, a reference value stored in storage unit 93 may relate to a non-healthy tissue, e.g., an abnormal tissue or a tissue having a pathology, such that analyzer 92 may process the sensed value in relation to a reference value of a non-healthy tissue. In some embodiments, analyzer 92 may perform an analysis in relation to multiple reference values, for example, to determine whether a sensed value is closer or better matches a reference value attributed to a healthy tissue, or whether a sensed value is closer or better matches a reference value attributed to a non-healthy tissue. For example, storage unit 93 may store a first reference value of 90 which may be attributed to a healthy tissue, and a second reference value of 40 which may be attributed to a non-healthy tissue. Analyzer 92 may determine or estimate a sensed value of 60, may determined that the sensed value is closer to the reference value of a non-healthy tissue than to the reference value of the healthy tissue, and may thereby determine and/or indicate a relatively higher probability that the sensed value is associated with a light reflected from a non-healthy tissue. In some embodiments, the reference value stored in storage unit 93 may include, for example, intensity values for one or more colors or a set of colors, e.g., a red intensity value, a green intensity value, a blue intensity value, or other suitable reference values.

In some embodiments, analyzer 92 may analyze light reflected from an area of interest of a tissue or body organ, which may partially or completely overlap with an area of interest imaged by imager 46. For example, imager 46 may image a first area of interest (“imaged area of interest”), and analyzer 92 may analyze light reflected from a second area of interest (“analyzed area of interest”). In one embodiment, the imaged area of interest and the analyzed area of interest may be substantially identical or may overlap, e.g., if analyzer 92 is integrated within imager 46. In another embodiment, the analyzed area of interest may partially overlap with the imaged area of interest. In yet another embodiment, the analyzed area of interest may be a portion of the imaged area of interest, e.g., a relatively small central portion.

In one embodiment, analyzer 92 may perform analysis operations substantially continuously and/or in real time. In another embodiment, analyzer 92 may perform analysis operations in accordance with pre-determined time periods, frequency, schedule, time intervals or time slots, e.g., one analysis process may be performed substantially every five seconds; other time intervals or frequencies may be used. In yet another embodiment, analyzer 92 may perform analysis operations upon demand, or in response to a signal, a request or a command from another component of device 40 or from another or external device. In still another embodiment, analyzer 92 may perform analysis operations when a triggering event occurs, or when one or more pre-defined condition or criteria are met.

In some embodiments, analyzer separator 91 and/or analyzer 92 may produce, and transmitter 41 may transmit, for example, color-separation data or separated-color data. In one embodiment, the data may include a color pattern or a “rainbow” pattern. In another embodiment, the data may include a value indicating a level of one or more colors, for example, produced by a color detector or a light detecting diode within device 40 (e.g., included in analyzer 92 or imager 46). In another embodiment, the data may include analysis results or diagnosis results, e.g., data indicating a healthy tissue, a non-healthy tissue, a tissue having an abnormality, a tissue having a pathology, or the like. Other suitable types of data may be produced and/or transmitted by device 40.

Although part of the discussion herein may relate, for exemplary purposes, to analysis and processing operations performed by analyzer 92, embodiments of the present invention are not limited in this regard. In some embodiments, analysis and processing operations may be performed by processor 47, by transmitter 47 having processing capabilities, by data processor 14, by other suitable components of device 40, or by other suitable components of the system of FIG. 1.

Although part of the discussion herein may relate, for exemplary purposes, to illumination, reflection and/or separation of a white light, embodiments of the present invention are not limited in this regard. In some embodiments, non-white light may be illuminated, reflected and/or separated; for example, the light being illuminated, reflected and/or separated may be a light having one pre-defined color (e.g., a blue color or a red color), may be a light having a combination or blend of two or more colors, or the like. Although portions of the discussion herein may relate, for demonstrative purposes, to optical analysis performed within the in-vivo device 40 (e.g., using the analyzer 92 of device 40), embodiments of the invention are not limited in this regard. For example, in some embodiments, an analyzer unit (e.g., substantially identical or similar to analyzer 92) may be located, in addition to or instead of within device 40, in a unit external to device 40 and/or external to the patient, for example, in receiver 12, in a workstation including the data processor 14, as a stand-alone external unit, or the like. For example, the external analyzer unit may receive in-vivo images, captured by imager 46, of light reflected from a body lumen or a body organ, the light rays passing through separator 91 prior to the image acquisition by imager 46; and the external analyzer unit may analyze such in-vivo images, e.g., similar to the analysis which may be performed in-vivo by analyzer 92 as detailed herein.

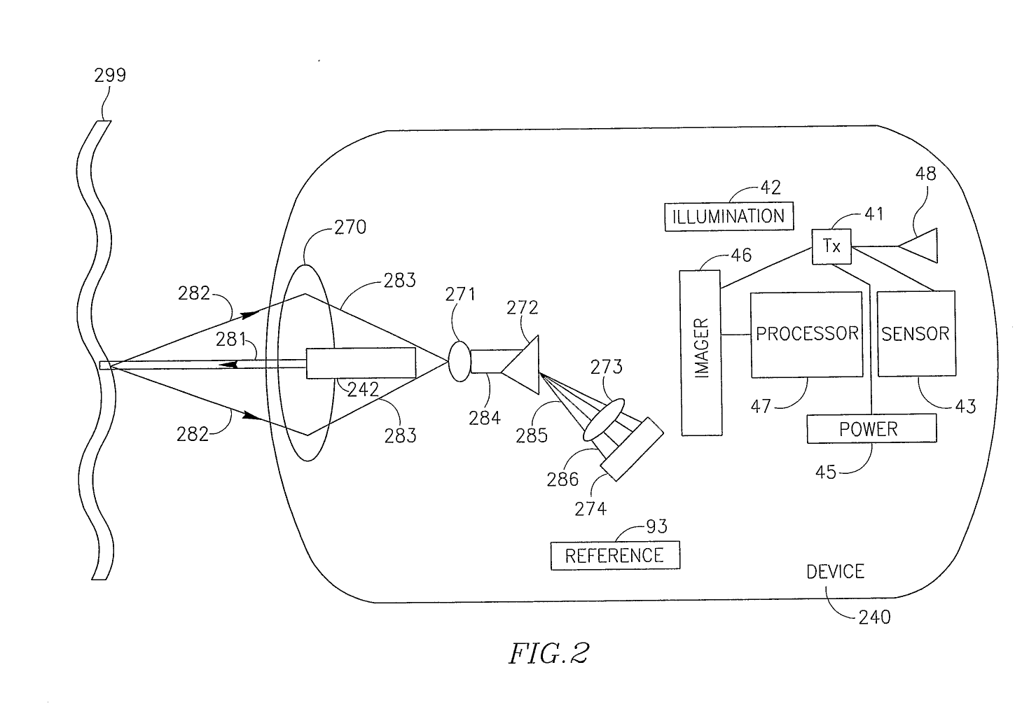

FIG. 2 shows a schematic diagram of an in-vivo imaging device 240 in accordance with an embodiment of the present invention. Device 240 may be an example of device 40 of FIG. 1. Device 240 may be used, for example, in conjunction with the in-vivo imaging system of FIG. 1 or other suitable in-vivo imaging systems.

Device 240 may include, for example, imager 46, one or more illumination sources 42, transmitter 41, antenna 48, power source 45, and optionally processor 47 and sensor 43. Device 240 may further include an illumination source 242 configured to illuminate white or other light, e.g., a “white LED” unit. For example, illumination unit 242 may generate a ray or beam 281 of white light, which may be illuminated towards a body organ or tissue 299 while device 240 is in-vivo. Body organ or tissue 299 may include, for example, a portion of the GI tract, and may include one or more tissue layers. Body organ or tissue 299 may reflect the ray or beam 281 of white light, generating rays or beams 281 of reflected light. Rays or beams 282 of the reflected light may include, for example, a flat collimate beam of reflected light.

Device 240 may further include an optical element 270 configured to receive the rays or beams 282 of the reflected light, e.g., a focusing lens. Optical element 270 may transfer or focus the rays or beams 282 of the reflected light into focused rays or beams 283 and onto a collimating lens 271, e.g., a cylinder collimating lens. Collimating lens 271 may receive the rays or beams 283 of focused light, which may include non-parallel rays or beams 283, and may generate substantially parallel rays or beams 284 which may be transferred to a separator 272, e.g., a prism or a grating. Prism 272 may receive the substantially parallel rays or beams 284, which may include white light reflected from tissue 299, and may separate or divide the white light into one or more colors or spectral elements, e.g., into color rays or beams 285.

Optionally, one or more color rays or beams 285 may pass through a color filter 273, e.g., to filter-in or filter-out one or more colors. For example, in one embodiment, color rays or beams 285 may pass through a filter 273 which may filter out non-blue rays or beams, thereby producing a substantially single-color light 286. Other single-color lights may be produced and used.

Device 240 may further include an analyzer 274, which may receive the single-color light 286, and may perform processing or analyzing operations. In one embodiment, analyzer 274 may include a dedicated detector or imager, e.g., a monochrome imager. In another embodiment, analyzer 274 may be part of, or may be integrated with, imager 46, transmitter 41 and/or processor 47. In some embodiments, analyzer 274 may be an example of analyzer 92 of FIG. 1, and/or may be included in data processor 14.

In some embodiments, analyzer 274 may process or analyze the single-color light 286, for example, in relation to pre-defined reference values which may be stored in reference value storage 93. As discussed above with reference to FIG. 1, the analysis may allow, for example, analyzer 274 to determine whether light reflected from tissue 299 indicates that tissue 299 is healthy, non-healthy, or includes a pathology or an abnormality.

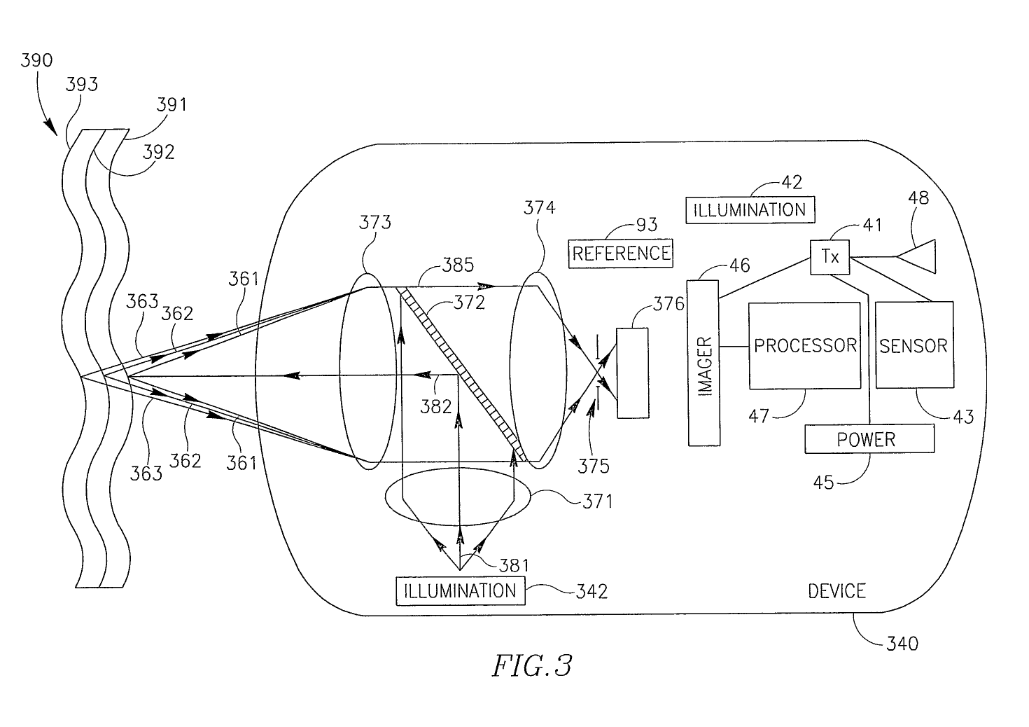

FIG. 3 shows a schematic diagram of an in-vivo imaging device 340 in accordance with an embodiment of the present invention. Device 340 may be an example of device 40 of FIG. 1. Device 340 may be used, for example, in conjunction with the in-vivo imaging system of FIG. 1 or other suitable in-vivo imaging systems.

Device 340 may include, for example, imager 46, one or more illumination sources 42, transmitter 41, antenna 48, power source 45, and optionally processor 47 and sensor 43. Device 340 may further include an illumination source 342 configured to illuminate white light, e.g., a “white LED” unit. For example, illumination unit 342 may generate rays or beams 381 of white light, which may be illuminated through a non-dispersive lens 371 towards a beam splitter, e.g., a semi-reflective mirror 372 at an angle of substantially 45 degrees. Mirror 372 may split or divide the rays or beams 381 of illuminated light, for example, such that substantially half of rays or beams 381 may be reflected back towards illumination unit 342, and substantially half of rays or beams 381 may be reflected from mirror 372 as rays or beams 382, e.g., towards a body organ or tissue 390 while device 340 is in-vivo. Body organ or tissue 390 may include, for example, a portion of the GI tract, and may include one or more tissue layers, for example, tissue layers 391, 392 and 393.

The rays or beams 382 of white light may include light having a plurality of colors, which may be reflected from one or more tissue layers 391-393. For example, first tissue layer 391 may reflect rays or beams 361, second tissue layer 392 may reflect rays of beams 362, and third tissue layer may reflect rays or beams 363. The rays or beams 361-363 reflected from body organ or tissue 390 may be received by a dispersive lens 373, which may, for example, produce substantially parallel rays or beams 385, which may be received by a non-dispersive lens 374. In some embodiments, dispersive lens 374 may focus the rays or beams 385 onto an analyzer 376, optionally through a hole, a pin hole or an aperture 375. In some embodiments, aperture 375 may perform spatial filtering of light, thereby allowing analyzer 376 to avoid detection or analysis of scattered light or stray light.

In some embodiments, lens 373 may be substantially parallel to lens 374, lens 371 may be substantially perpendicular to lenses 373 and 374, and semi-reflective mirror 372 may be positioned at an angle of substantially 45 degrees to lens 371 and/or lens 373. In some embodiments, the optical arrangement of illumination unit 342, non-dispersive lens 371, semi-reflective mirror 372, dispersive lens 373, non-dispersive lens 374, aperture 375 and/or analyzer 376 may be similar to a Michelson Interferometer arrangement, such that an interference may occur and an interference pattern may appear, for example, between one or more rays of beams reflected back from tissue 390 and one or more rays or beams reflected by mirror 372 towards illumination unit 342. In some embodiments, the interference pattern may be imaged, processed and/or analyzed, for example, by analyzer 376.

For example, analyzer 376 may receive light having an interference pattern (“sensed pattern”) and may compare it, or analyze it in relation to, pre-defined reference values of an interference pattern (“reference pattern”). In some embodiments, for example, reference pattern values may be stored in reference storage unit 93, and analyzer 376 may compare the sensed pattern values to the stored reference pattern values. As discussed above with reference to FIG. 1, the analysis may allow, for example, analyzer 376 to determine whether light reflected from tissue 390 indicates that tissue 390 or layers 391-393 are healthy, non-healthy, or include a pathology or an abnormality.

In one embodiment, analyzer 376 may include a dedicated detector or imager, e.g., a monochrome imager. In another embodiment, analyzer 376 may be part of, or may be integrated with, imager 46, transmitter 41 and/or processor 47. In some embodiments, analyzer 376 may be an example of analyzer 92 of FIG. 1.

In some embodiments, analyzer 376 or other components of device 340 may use one or more other algorithms for processing or analyzing reflected light, for example, an Optical Coherence Tomography (OTC) algorithm.

It is noted that in some embodiments, lens 373 or other optical elements in device 340 may include a dispersive lens, able to focus rays of light having different colors at different distances, thereby allowing separation of reflected light into colors, for further analysis by analyzer 376.

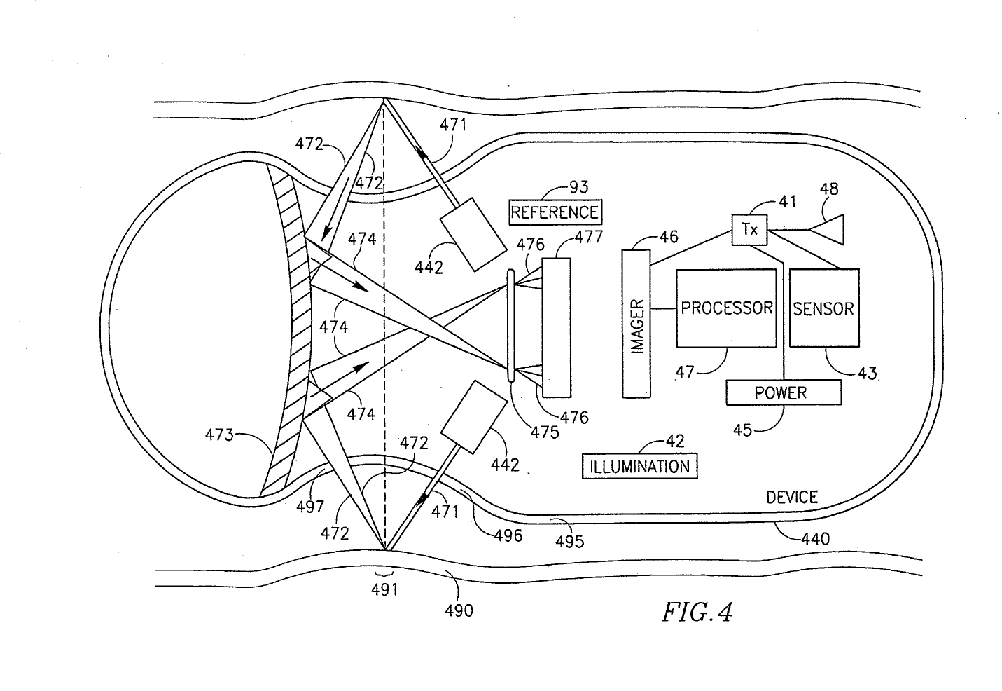

FIG. 4 shows a schematic diagram of an in-vivo imaging device 440 in accordance with an embodiment of the present invention. Device 440 may be an example of device 40 of FIG. 1. Device 440 may be used, for example, in conjunction with the in-vivo imaging system of FIG. 1 or other suitable in-vivo imaging systems.

Device 440 may include, for example, imager 46, one or more illumination sources 42, transmitter 41, antenna 48, power source 45, and optionally processor 47 and sensor 43. Device 440 may further include one or more illumination sources 442 configured to illuminate white light, e.g., a plurality of “white LED” unit. In some embodiments, illumination sources 442 may be arranged in a ring-shaped arrangement, an oval arrangement or a circular arrangement. Device 440 may be inside a body lumen 490, and illumination sources 442 may be arranged as a ring and may illuminate rays or beams 471 towards body lumen 490, such that a ring-shaped portion 491 of body lumen 490 may be illuminated by the white light of illumination sources 442. In some embodiments, portion 491 of body lumen 490 may reflect the illuminated rays or beams 471 as reflected rays or beams 472, which may be received by a curved optical element, e.g., a convex or panoramic mirror 473. It is noted that in some embodiments, body lumen 490 may include one or more tissue layers, which may reflect different colors of the illuminated light at different angles or patterns.

Mirror 473 may receive the rays or beams 472 reflected from body lumen 490, and may reflect them as rays or beams 474. In some embodiments, rays or beams 472 may be widening or dispersive, whereas rays or beams 474 may be narrowing or converging.

It is noted that in some embodiments, device 440 may include a transparent or semi-transparent housing 495, which may allow transfer of light from device 440 to its vicinity, or vice versa. In some embodiments, housing 495 may be shaped to facilitate transfer of light, for example, such that housing 495 may have a portion 496 which may be substantially perpendicular to rays or beams 471, or a portion 497 which may be substantially perpendicular to at least some of rays or beams 472.

Rays or beams 474 may be received by a separator 475, e.g., a grating or a prism, which may separate rays or beams 474 into color rays or beams 476. An analyzer unit 477 may receive the color rays or beams 476, and may process or analyze them.

In one embodiment, analyzer 477 may include a dedicated detector or imager, e.g., a monochrome imager or a multi-color imager. In another embodiment, analyzer 477 may be part of, or may be integrated with, imager 46, transmitter 41 and/or processor 47. In some embodiments, analyzer 477 may be an example of analyzer 92 of FIG. 1.

In some embodiments, the dispersion of ring-shaped white light illuminated onto portion 491 of body lumen 490, may be reflected such that different colors are reflected at different angles by different tissue layers of body lumen 490. This may allow analyzer 477, for example, to receive and analyze reflection data indicating one or more properties of body lumen 490. In some embodiments, for example, analyzer 477 may receive a plurality of colored ring-shaped patterns or images, which may be created from the reflected white ring of light separated by separator 475; for example, a blue ring may be surrounded by a red ring, or vice versa, or other colored rings or “rainbow” patterns may be detected by analyzer 477.

In some embodiments, analyzer 477 may receive panoramic or circular colored images (“sensed panoramic images”) and may compare them to, or analyze them in relation to, pre-defined reference values of circular colored images (“reference panoramic images”) or color data. In alternate embodiments, analyzer 477 may receive the sensed panoramic images, and may separate, separate-out or filter-out one or more colors, and device 40 may transmit color data, separated-color date or filtered-color data.

In some embodiments, for example, reference panoramic images values may be stored in reference storage unit 93, and analyzer 477 may compare the sensed panoramic images values to the stored reference panoramic images values. As discussed above with reference to FIG. 1, the analysis may allow, for example, analyzer 477 to determine whether light reflected from tissue 490 indicates that tissue 490 is healthy, non-healthy, or includes a pathology or an abnormality.

Device 440, or other in-vivo devices or systems in accordance with embodiments of the present invention, may include other suitable optical elements or other suitable components, for example, in addition to or instead of convex or panoramic mirror 473. For example, some embodiments of the invention may be used in conjunction with, or may include or otherwise incorporate, devices, systems, methods or components as described in U.S. patent application Ser. No. 10/836,614, entitled “Endoscope with Panoramic View”, filed on May 3, 2004, which is incorporated herein by reference in its entirety; some embodiments of the invention may be used in conjunction with, or may include or otherwise incorporate, devices, systems, methods or components as described in U.S. patent application Ser. No. 10/879,284, entitled “Device, System, and Method for Reducing Image Data Captured In-Vivo”, filed on Jun. 30, 2004, which is incorporated herein by reference in its entirety; some embodiments of the invention may be used in conjunction with, or may include or otherwise incorporate, devices, systems, methods or components as described in International Patent Application Number PCT/IL2004/000367, entitled “Panoramic Field of View Imaging Device”, filed on May 2, 2004, Published on Nov. 11, 2004 as International Publication Number WO 2004/096008, which is incorporated herein by reference in its entirety; and some embodiments of the invention may be used in conjunction with, or may include or otherwise incorporate, other suitable devices, systems, methods or components.

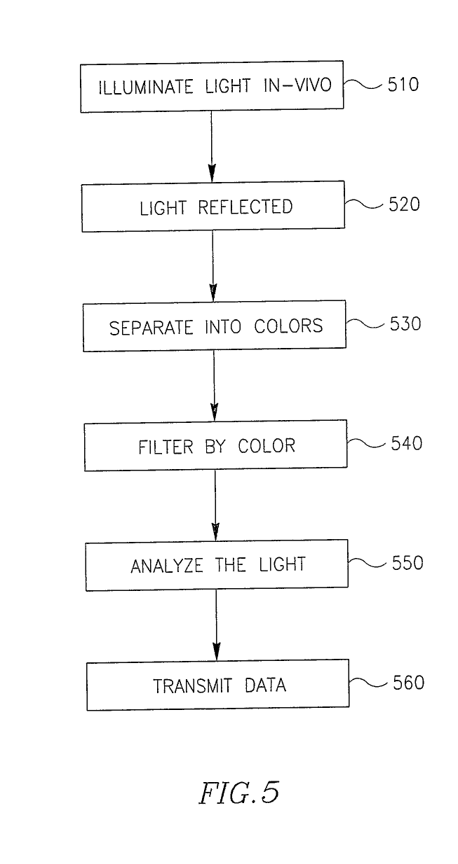

FIG. 5 is a flow-chart diagram of a method of optical biopsy in accordance with an embodiment of the present invention. The method of FIG. 5, as well as other suitable methods in accordance with embodiments of the invention, may be used, for example, in association with the system of FIG. 1, with device 40 of FIG. 1, with device 240 of FIG. 2, with device 340 of FIG. 3, with device 440 of FIG. 4, with one or more in-vivo imaging devices (which may be, but need not be, similar to device 40), and/or with other suitable devices and systems for in-vivo imaging. A method according to some embodiments of the invention need not be used in an in-vivo context.

In some embodiments, as indicated at box 510, light may be illuminated in-vivo on a body lumen or tissue. For example, substantially white light may be illuminated.

As indicated at box 520, the illuminated light may be reflected from the body organ or tissue. For example, a multi-layer tissue may reflect the light such that different tissue layers may reflect different colors of light at different angles or patterns.

As indicated at box 530, the reflected light may be separated or filtered into colors, spectra, bandwidths or wavelengths, e.g., using a prism, a grating, a color separator, a color divider, one or more filters, or other suitable optical components.

As indicated at box 540, the separated light may optionally be filtered, e.g., using a color filter which may filter-in or filter-out one or more pre-defined colors.

As indicated at box 550, the separated light may be analyzed or processed. In one embodiment, this may be performed in relation to one or more pre-defined reference values which may correlate to one or more values obtained from illuminating a healthy tissue, a non-healthy tissue, or a tissue having an abnormality or pathology. In some embodiments, the analysis may be performed in-vivo and/or substantially in real time.

As indicated at box 560, optionally, analysis results may be transmitted, e.g., from the in-vivo imaging device towards an external receiver. Optionally, the transmission may include image data, or separated-color image data.

It is noted that some or all of the above-mentioned operations may be performed substantially in real time, e.g., during the operation of the in-vivo imaging device, during the time in which the in-vivo imaging device operates and/or captures images, and/or without interruption to the operation of the in-vivo imaging device. In some embodiments, some or all of the above-mentioned operations may be performed while images are acquired, transmitted and/or recorded, or after one or more, or substantially all, images are acquired, transmitted and/or recorded.

Other operations or sets of operations may be used in accordance with embodiments of the invention.

A device, system and method in accordance with some embodiments of the invention may be used, for example, in conjunction with a device which may be inserted into a human body or swallowed by a person. However, the scope of the present invention is not limited in this regard; for example, some embodiments of the invention may be used in conjunction with a device which may be inserted into, or swallowed by, a non-human body or an animal body.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

1. An in-vivo imaging device comprising:
a separator configured to separate a light, reflected from a body lumen, into at least one separated-color light.

2. The in-vivo imaging device of claim 1, wherein said separator is selected from the group consisting of: a prism, a grating, a Michelson Interferometer arrangement and a color filter.

3-5. (canceled)

6. The in-vivo imaging device of claim 1, comprising an analyzer configured to analyze said at least one separated-color light.

7. The in-vivo imaging device of claim 6, wherein said analyzer is configured to analyze an interference pattern

8. The in-vivo imaging device of claim 6, wherein said analyzer is configured to analyze said at least one separated-color light in relation to a pre-defined reference value.

9. The in-vivo imaging device of claim 6, wherein said analyzer is configured to determine if said at least one separated-color light is reflected from a healthy tissue.

10. The in-vivo imaging device of claim 6, wherein said analyzer is configured to determine if said at least one separated-color light is reflected from a non-healthy tissue.

11. The in-vivo imaging device of claim 1, comprising:
an illumination unit to illuminate a white light through a non-dispersive lens;
a semi-reflective mirror to split said white light into substantially a first half and a second half, said first half illuminated onto said body lumen, and said second half reflected back towards said illumination unit;
a dispersive lens to receive said first half reflected from said body lumen; and
a non-dispersive lens to focus an interference pattern of said first half transferred through said dispersive lens and said second half.

12. The in-vivo imaging device of claim 1, wherein said separator comprises:
a panoramic mirror to receive and reflect the light reflected from said body lumen; and
a grating to separate the light reflected from said panoramic mirror into said at least one separated-color light.

13. The in-vivo imaging device of claim 12, comprising an illumination unit to illuminate a substantially ring-shaped dispersion of white light towards said body lumen.

14. The device of claim 1, wherein said device is a swallowable capsule.

15. The device of claim 1, wherein said device is an autonomous in-vivo device.

16. The device of claim 1, comprising a light detector.

17. The device of claim 1, comprising a color detector.

18. The device of claim 1, comprising an imager.

19. The device of claim 6, comprising a transmitter to transmit an analysis result produced by said analyzer.

20. A method comprising:
separating in-vivo a light, reflected from a body lumen, into at least one separated-color light.

21. The method of claim 20, comprising analyzing said at least one separated-color light.

22. The method of claim 20, comprising analyzing in-vivo said at least one separated-color light.

23. The method of claim 20, comprising analyzing said at least one separated-color light in relation to a reference value.

24. The method of claim 20, comprising filtering said at least one separated-color light.

25. The method of claim 21, comprising determining whether said body lumen comprises one chosen from the group consisting of: a non-healthy body lumen, an abnormality and a pathology.

26-31. (canceled)

32. The in-vivo imaging device of claim 8, wherein said pre-defined reference value is stored within said in-vivo imaging device.

33-34. (canceled)

35. The device of claim 6, wherein said analyzer is external to the in-vivo device.

36. A system for in vivo imaging comprising said in vivo imaging device of claim 6, and a receiver to receive an in-vivo image from the in-vivo imaging device, and to transfer the in-vivo image to said analyzer.
